(19) United States
(12) Patent Application Publication
(10) Pub. No.: US 2014/0261409 A1
(43) Pub. Date: Sep. 18, 2014

(54) SYSTEMS AND METHODS FOR VENTILATION WITH UNRELIABLE EXHALATION FLOW AND/OR EXHALATION PRESSURE

(71) Applicant: COVIDIEN LP, Boulder, CO (US)

(72) Inventors: Nancy F. Dong, Carlsbad, CA (US); Richard Nakai, Long Beach, CA (US); Hari Damineni, San Diego, CA (US); Gabriel Sanchez, Valley Center, CA (US)

(73) Assignee: Covidien LP, Boulder, CO (US)

(21) Appl. No.: 13/798,584

(22) Filed: Mar. 13, 2013

(51) Int. Cl. A61M 16/00; A61M 16/04; A61M 16/06; A61M 16/08

(52) U.S. Cl. CPC A61M 16/0051; A61M 16/0057; A61M 16/0875; A61M 16/04; A61M 16/06
USPC 128/202.22; 128/204.23

(57) ABSTRACT

This disclosure describes systems and methods for providing novel back-up ventilation that allows the patient to trigger or initiate the delivery of a breath. Further, this disclosure describes systems and methods for triggering ventilation when exhalation flow and/or exhalation pressure is unknown or unreliable by the ventilator.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes or settings have been created to provide better ventilation for patients in various different scenarios, such as mandatory ventilation modes and assist control ventilation modes.

Ventilation with Unreliable Exhalation Flow and/or Exhalation Pressure

This disclosure describes systems and methods for providing novel back-up ventilation that allows the patient to trigger or initiate the delivery of a breath. Further, this disclosure describes systems and methods for triggering ventilation when exhalation flow and/or exhalation pressure is unknown or unreliable by the ventilator.

In part, this disclosure describes a method for ventilating a patient with a ventilator. The method includes:

a) delivering a fixed base flow;

b) monitoring inspiratory pressure and exhalation flow during ventilation of a patient with a ventilator;

c) delivering ventilation based at least on the monitored exhalation flow;

d) triggering inspiration during the ventilation based at least on the monitored exhalation flow based on the first of at least one of the following events to occur: detecting a first trigger condition; and detecting expiration of a predetermined amount of time;

e) determining a malfunction that makes the monitored exhalation flow unreliable; and in response to the malfunction: ceasing ventilation based on the monitored exhalation flow; estimating an exhalation flow based on the monitored inspiratory pressure; delivering ventilation based at least on the estimated exhalation flow; triggering inspiration during the ventilation based at least on the estimated exhalation flow based on the first of at least one of the following events to occur: detecting a second trigger condition based at least on the estimated exhalation flow; and detecting expiration of the predetermined amount of time.

Yet another aspect of this disclosure describes a ventilator system that includes: a pressure generating system adapted to generate a flow of breathing gas including a fixed base flow; a ventilation tubing system including a patient interface for connecting the pressure generating system to a patient; an exhalation valve connected to the ventilation tubing system; a plurality of sensors operatively coupled to at least one of the pressure generating system, the patient, and the ventilation tubing system for monitoring inspiratory pressure, inspiratory flow, exhalation pressure, and exhalation flow; an exhalation flow estimation module, the exhalation flow estimation module estimates exhalation flow based on the monitored inspiratory pressure; a main driver, the main driver controls the exhalation valve to deliver ventilation to the patient based at least on at least one of the exhalation pressure and the exhalation flow monitored by the plurality of sensors; a main trigger module, the main trigger module triggers an inspiration based on the first of at least one of the following events to occur: detection of a first trigger condition, and expiration of a predetermined amount of time; a backup driver, the backup driver controls the exhalation valve to deliver the ventilation to the patient based on at least one of the inhalation pressure and the inhalation flow monitored by the plurality of sensors; a backup trigger module, the backup trigger module triggers the inspiration based on the first of at least one of the following events to occur: detection of a second trigger condition based at least on the estimated exhalation flow, and expiration of the predetermined amount of time; and a controller, the controller determines a malfunction that makes the monitored exhalation flow unreliable and switches from the main driver and the main trigger module to the backup driver and the backup trigger module.

The disclosure further describes a computer-readable medium having computer-executable instructions for performing a method of ventilating a patient with a ventilator. The method includes:

a) repeatedly delivering a fixed base flow;

b) repeatedly monitoring inspiratory pressure and exhalation flow during ventilation of a patient with a ventilator;

c) repeatedly delivering ventilation based at least on the monitored exhalation flow;

d) repeatedly triggering inspiration during the ventilation based at least on the monitored exhalation flow based on the first of at least one of the following events to occur: detecting a first trigger condition; and detecting expiration of a predetermined amount of time;

e) determining a malfunction that makes the monitored exhalation flow unreliable; and in response to the malfunction: ceasing ventilation based on the monitored exhalation flow; repeatedly estimating an exhalation flow based on the monitored inspiratory pressure; repeatedly delivering ventilation based at least on the estimated exhalation flow; repeatedly triggering inspiration during the ventilation based at least on the estimated exhalation flow based on the first of at least one of the following events to occur: detecting a second trigger condition based at least on the estimated exhalation flow; and detecting expiration of the predetermined amount of time.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims.

FIG. 1A is a diagram illustrating an embodiment of an exemplary ventilator.

FIG. 1B illustrates an embodiment of the ventilator shown in FIG. 1A.

FIG. 2 illustrates an embodiment of a method for triggering inspiration during ventilation of a patient on a ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

As each patient may require a different ventilation strategy, modern ventilators can be customized for the particular needs of an individual patient. For example, several different ventilator modes or settings have been created to provide better ventilation for patients in various different scenarios, such as mandatory ventilation modes and assist control ventilation modes. Assist control modes allow a spontaneously breathing patient to trigger inspiration during ventilation.

The response performance of a medical ventilator to a patient trigger from exhalation into inhalation phase represents an important characteristic of a medical ventilator. A ventilator's trigger response impacts the patient's work of breathing and the overall patient-ventilator synchrony. The trigger response performance of a ventilator is a function of a patient's inspiratory behavior (breathing effort magnitude and timing characteristics) as well as the ventilator's gas delivery dynamics and flow control parameters (actuator response, dead bands, etc.).

In conventional flow triggering modes, a patient's inspiratory trigger is detected based on the magnitude of flow deviations generated by the patient's inspiratory effort. In a flow triggering mode, the ventilator delivers a fixed base flow during the exhalation phase. Accordingly, flow deviations are sensed by the computation of the ventilator net flow (base flow-exhausted flow) and compared against a set trigger threshold for triggering. As used herein, a trigger condition is met when a situation occurs that should trigger the delivery of a breath. For example, a trigger condition is met when a trigger threshold is breached, or exceeded, a predetermined amount of time has expired, and/or exhalation flow becomes stable.

Base flow is the delivered flow during exhalation and consists of a desired combination of appropriate gases. A fixed base flow may be generated by a controller regulating an actuator (valve) to maintain a constant desired flow rate from a regulated pressurized gas source into the ventilator circuit. The magnitude or the flow rate generated by the regulator at different open positions is determined by an inspiratory flow sensor. Therefore, base flow is determined by the ventilator by measuring the amount of flow delivered to the patient via an inspiration flow sensor during exhalation.

Exhausted flow is measured during the expiratory phase of a ventilator breath while a base flow is delivered through the patient circuit. To determine the volume of gas exhaled by the patient, the net flow (total delivered flow minus total flow through exhalation module) is used for integration. That is, the delivered base flow is subtracted from the sum of the base flow and patient flow exiting through the exhalation port. The flow exiting the exhalation module during the active phase of patient exhalation is the sum of base flow delivered by the ventilator and exhaled flow from the patient lung.

In the event of malfunctions and/or system failures in ventilators, ventilators, typically, sound an alarm and stop ventilation. Ventilators stop ventilation because the necessary parameters for delivering the desired ventilation are unreliable or undeterminable due to the malfunction.

For example, the ventilator utilizes several systems and/or components to control the spontaneous triggering of the delivery of a breath to the patient, such as the source of gas, the inspiratory conduit and valve, the inspiratory module, exhalation conduit and valve, an exhalation module, and a controller. The expiratory module utilizes measured expiratory flow and/or expiratory pressure to control the exhalation valve to deliver the desired amount of flow and/or pressure during inspiration and exhalation. For example, the controller controls when to deliver inspiration based on spontaneous effort from the patient which can be determined by exhalation flow and/or exhalation pressure. If exhalation flow and/or exhalation pressure are unavailable, the ventilator is unable to determine when to trigger delivery of breath to the patient and therefore ceases ventilation. However, it is desirable to provide ventilation to a patient whose ability to breathe on his or her own is impaired. Accordingly, the systems and methods disclosed herein provide ventilation in the event that exhalation pressure and/or exhalation flow are undeterminable.

In the absence of an exhalation flow sensor, under fault conditions, or during a malfunction of the expiratory system, the exhalation flow sensor and the exhalation pressure sensor are unreliable. Therefore, monitored exhalation flow and/or monitored exhalation pressure are unreliable or undeterminable, so a conventional flow triggering algorithm cannot be used to compare the net flow (base flow−exhausted flow) against the trigger threshold. Accordingly, patient initiated triggers cannot be detected by previously utilized ventilators and prevented the use of a spontaneous mode of ventilation in these ventilators. However, the systems and methods as described herein utilize monitored inspiratory pressure and/or monitored inspiratory flow to estimate an exhalation flow.

An example of a fault condition is presented by the Exhalation Back-Up Ventilation (EBUV) mode under which the data measurement and acquisition subsystem on the exhalation side of the ventilator is deactivated because of a malfunction. As discussed above, conventional ventilators declare an alarm and terminate ventilation. However, the EBUV mode allows a ventilator to continue ventilating the patient under such conditions, thereby maintaining a reduced work of breathing and increased patient-ventilator synchrony when compared to conventional ventilators, until an appropriate substitute device is made available.

Accordingly, the systems and methods described herein provide for a triggering mechanism when an exhalation flow and/or exhalation pressure is undeterminable by the ventilator. For example, the exhalation flow and/or exhalation pressure is undeterminable by the ventilator when a malfunction is detected in the exhalation flow sensor, exhalation pressure sensor, exhalation valve command, and/or any other sensor and/or module relevant to exhalation flow are malfunctioning. The capability of triggering without the exhalation flow and/or exhalation pressure allows an EBUV mode to maintain comfortable patient-ventilator synchrony. The systems and methods described herein provide a triggering mechanism for a spontaneous patient when the ventilator cannot determine the exhalation flow. The ventilator estimates an exhalation flow based on monitoring inspiratory pressure and/or inspiratory flow. The estimated exhalation flow is substituted for the actual exhalation flow allowing the traditional flow triggering algorithm to be utilized. For example, the ventilator is able to determine flow deviations by the computation of the ventilator net flow (base flow-estimated exhausted flow) which is compared against a set trigger threshold for triggering.

FIGS. 1A and 1B are diagrams illustrating an embodiment of an exemplary ventilator 100. The exemplary ventilator 100 illustrated in FIG. 1A is connected to a human patient 150. The ventilator 100 includes a pneumatic system 102 (also referred to as a pressure generating system 102) for circulating breathing gases to and from the patient 150 via a ventilation tubing system 130, which couples the patient 150 to the pneumatic system 102 via an invasive (e.g., endotracheal tube, as shown) or a non-invasive (e.g., nasal mask) patient interface 180. The pneumatic system 102 delivers ventilation to the patient 150 according to predetermined or selected modes (spontaneous, assist, mandatory, etc.) and breath types (pressure control, pressure support, pressure assist, volume control, volume support, volume-controlled-pressure-targeted, etc.).

The ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple the patient interface 180 (shown as an endotracheal tube in FIG. 1A and as a nasal mask in FIG. 1B) to an inspiratory limb 132 and an exhalation limb 134 of the ventilation tubing system 130.

The pneumatic system 102 may be configured in a variety of ways. In the present example, the pneumatic system 102 includes an exhalation module 108 coupled with the exhalation limb 134 and an inspiratory module 104 coupled with the inspiratory limb 132. A compressor 106, accumulator 124 (as illustrated in FIG. 1B) and/or other source(s) of pressurized gases (e.g., air, oxygen, and/or helium) is coupled with the inspiratory module 104 and the exhalation module 108 to provide a gas source for ventilatory support via the inspiratory limb 132. In an embodiment, the pneumatic system 102 is operatively coupled with, and at times receives directions from, a controller 110.

The inspiratory module 104 is configured to deliver gases to the patient 150 and/or through the inspiratory limb 132 according to prescribed ventilatory settings. The inspiratory module 104 is associated with and/or controls an inspiratory delivery valve 101 for controlling gas delivery to the patient 150 and/or gas delivery through the inspiratory limb 132 as illustrated in FIG. 1B. In some embodiments, the inspiratory module 104 is configured to provide ventilation according to various ventilator modes, such as mandatory and assist modes.

The exhalation module 108 is configured to release gases from the patient's lungs and/or exhalation circuit according to prescribed ventilatory settings. Accordingly, the exhalation module 108 also controls gas delivery through the inspiratory limb 132 and the exhalation limb 134. The exhalation module 108 controls an exhalation valve 123 to control gases from the patient's lungs and/or exhalation circuit according to prescribed ventilatory settings.

The ventilator 100 includes a main driver 103 for controlling the exhalation valve 123. In some embodiments, the main driver 103 is part of the exhalation module 108. In other embodiments the main driver 103 is included in a different system or module, such as the pneumatic system 102. The main driver 103 controls the exhalation valve 123 to relieve the over pressure delivered during inhalation to deliver the desired inspiration pressure. Further, the main driver 103 controls the exhalation valve 123 to deliver the desired PEEP during exhalation. The main driver 103 is used by a control algorithm that is computed by utilizing monitored exhalation pressure and monitored exhalation flow. The monitored exhalation flow and/or pressure are determined by one or more of a plurality of sensors 107, which are discussed in further detail below.

In some embodiments, the main driver 103 is a differential driver. In other embodiments, the main driver 103 is a pulse width modulation driver. The above listed drivers are not meant to be limiting. Any suitable driver for controlling an exhalation module 108 in a ventilator may be utilized by the ventilator 100.

The ventilator 100 also includes a main trigger module 113 that triggers inspiration according to prescribed ventilatory settings. In some embodiments, as illustrated in FIG. 1A, the main trigger module 113 is included in a controller 110. In other embodiments the main trigger module 113 is included in a different system or module, such as the pneumatic system 102. In an embodiment, the main trigger module 113 triggers an inspiration based on the first of at least two events, such as the expiration of a predetermined amount of time and detection of a first trigger condition.

There are several different trigger types or systems and/or methods utilized by the ventilator 100 for detecting a first trigger condition. In some embodiments, a trigger type for detecting patient effort may be selected or input by an operator. In some embodiments, the trigger type is automatically selected by the ventilator. Any suitable type of triggering detection for determining a patient trigger may be utilized by the ventilator, such as nasal detection, diaphragm detection, and/or brain signal detection. Further, the ventilator may detect patient triggering via a pressure-monitoring method, a flow-monitoring method, direct or indirect measurement of neuromuscular signals, or any other suitable method. Sensors 107 suitable for this detection may include any suitable sensing device as known by a person of skill in the art for a ventilator. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight drop in circuit pressure. The slight drop in circuit pressure may indicate that the patient's respiratory muscles are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in baseline flow (or base flow) attributable to a slight redirection of gases into the patient's lungs (in response to a slightly negative pressure gradient as discussed above). Base flow refers to a constant flow existing in the circuit during exhalation that enables the ventilator to detect expiratory flow changes and patient triggering. For example, while gases are generally flowing out of the patient's lungs during exhalation, a drop in flow may occur as some gas is redirected and flows into the lungs in response to the slightly negative pressure gradient between the patient's lungs and the body's surface. Thus, when the ventilator detects a slight drop in flow below the base flow by a predetermined threshold amount (e.g., 2 L/min below base flow), it may interpret the drop as a patient trigger and may consequently initiate inspiration by delivering respiratory gases.

In one embodiment, the ventilator 100 is preconfigured to deliver an inspiration after a predetermined amount of exhalation time to prevent the patient 150 from becoming under-ventilated. Accordingly, the predetermined amount of exhalation time (e.g., known as an apnea interval in some ventilators) is the trigger threshold in this embodiment. For example, the main trigger module 113 will automatically trigger an inspiration after 20 seconds, 30 seconds, or 60 seconds of exhalation time. In some embodiments, the predetermined amount of time is determined by the clinician and/or ventilator 100 based on whether the patient 150 is an infant, child, adult, male, female, and/or suffering from a specific disease state.

The ventilator 100 includes a flow estimation module 117 that estimates an exhalation flow when a malfunction detected by the controller 110 establishes the monitored exhalation flow and/or the monitored exhalation pressure as undeterminable or unreliable. In some embodiments, as illustrated in FIG. 1A, the controller 110 includes the flow estimation module 117. In other embodiments, the pneumatic system 102 includes the flow estimation module 117. The terms “undeterminable” and “unreliable”, while having different meanings, are utilized interchangeably herein. Accordingly, the term “unreliable” encompasses the term “undeterminable” and the term “undeterminable” encompasses “unreliable.” In previous systems, if a malfunction was present, a ventilator could no longer deliver spontaneous ventilation. In order to provide spontaneous ventilation, the ventilator 100 estimates exhalation flow based on the monitored inspiratory flow and/or the monitored inspiratory pressure as determined by one or more of the plurality of sensors 107. Since an exhalation flow is estimated, the ventilator 100 may continue to spontaneously ventilate, maintaining patient-ventilator synchrony and/or patient comfort. In some embodiments, the flow estimation module 117 is a part of the exhalation module 108 and is communicatively coupled to an inspiratory flow sensor 109a and/or an inspiratory pressure sensor 109b. In other embodiments, the flow estimation module 117 is a part of the inspiratory module 104. The flow estimation module 117 is communicatively coupled to a backup trigger module 115, and/or the backup driver 105.

The ventilator 100 includes a backup driver 105 for controlling the exhalation valve 123. In some embodiments, the backup driver 105 is part of the exhalation module 108. In other embodiments, the backup driver 105 is included in a different system or module, such as the pneumatic system 102. The backup driver 105 controls the exhalation valve 123 to relieve the over pressure delivered during inhalation to deliver the desired inspiration pressure. Further, the backup driver 105 controls the exhalation valve 123 to deliver the desired PEEP during exhalation. Because the exhalation flow and/or exhalation pressure is not determinable, the amount of PEEP delivered is determined based on the monitored inspiration pressure and monitored inspiration flow during a malfunction. The backup driver 105 is used by an inspiration control algorithm to deliver the desired inspiration pressure that is computed by utilizing monitored inspiration pressure and monitored inspiration flow. The backup driver 105 is used by an exhalation control algorithm to deliver the PEEP that is computed by utilizing monitored inspiration pressure and monitored inspiration flow. In some embodiments, the exhalation control algorithm subtracts the measured inspiration pressure from the desired PEEP. The monitored inspiration flow and/or inspiration pressure are determined by one or more of the plurality of sensors 107.

In some embodiments, as illustrated in FIG. 1B, the backup driver 105 is on a backup circuit 105a that is separated from or isolated from the main driver 103 and the main driver circuit 103a. The main driver circuit 103a of the main driver 103 is connected to the exhalation valve 123 and one or more expiratory sensors, such as an expiratory flow sensor 111a and an expiratory pressure sensor 111b as illustrated in FIG. 1B. Additionally, the backup driver 105 is on a separate backup circuit 105a that connects the backup driver 105 to the exhalation valve 123 and is separated/isolated from an exhalation sensor (exhalation pressure sensor 111b and/or exhalation flow sensor 111a) and/or the main driver 103. A separate backup driver 105 and an isolated backup circuit 105a for the backup driver 105 allow the backup driver 105 to function regardless of a malfunctioning exhalation sensor and/or a malfunctioning main driver 103.

In some embodiments, the backup driver 103 is a pulse modulated driver. In other embodiments, the backup driver 105 is a pulse width modulation driver. The above listed drivers are not meant to be limiting. Any suitable driver for controlling an exhalation module 108 in a ventilator may be utilized by the ventilator 100.

Further, in some embodiments, the ventilator 100 includes a backup trigger module 115. In some embodiments, the backup trigger module 115 triggers inspiration according to prescribed ventilatory settings while the ventilator is in the EBUV mode. The controller 110 utilizes the backup trigger module 115 when a malfunction in the expiratory system is detected by the ventilator 100 or a subsystem of the ventilator, such as the controller 110. The malfunction prevents the monitored exhalation flow from being determined. In some embodiments, as illustrated in FIG. 1A, the controller 110 includes the backup trigger module 115. In other embodiments, the pneumatic system 102 includes the backup trigger module 115.

The backup trigger module 115 triggers inspiration based on the first of at least two events, such as the expiration of a predetermined amount of time and the detection of a second trigger condition. In some embodiments, the second trigger condition is a trigger threshold based on a flow deviation. In another embodiment, the second trigger condition is an inspiratory trigger threshold. In an embodiment, the backup trigger module 115 utilizes a fixed base flow, such as but not limited to 1.5 LPM, delivered by the pneumatic system 102 and an estimated exhalation flow, estimated by the flow estimation module 117, to determine a flow deviation, or net flow, which is compared to the trigger threshold. If the flow deviation breaches the trigger threshold then the controller 110 instructs the pneumatic system 102 to deliver a breath.

In some embodiments, the flow deviation is determined by adding or subtracting one of the fixed base flow and the estimated exhalation flow from the other. Because the exhalation flow is not determinable, an estimated exhalation flow is used to determine a flow deviation to be compared to the trigger threshold. The use of estimated exhalation flow allows the ventilator to continue triggering spontaneous breaths for the patient therefore maintaining patient-ventilator synchrony and patient comfort. The estimated exhalation flow is determined by the flow estimation module 117. In an embodiment, if the backup trigger module 115 determines that ventilator and/or patient parameters meet and/or exceed an inspiration trigger threshold during exhalation, the backup trigger module 115 instructs the inspiratory module 104 to deliver an inspiration, which effectively ends the exhalation phase. In another embodiment the backup trigger module 115 is included in a different system such as the pneumatic system 102.

If the backup trigger module 115 determines that ventilator and/or patient parameters do not meet and/or exceed an inspiration trigger threshold during exhalation, the backup trigger module 115 continues to monitor the ventilator and/or patient parameters and compare them to a trigger threshold until the ventilator and/or patient parameters meet and/or exceed a trigger threshold or until the expiration of a predetermined amount of time. If a trigger threshold is not breached within a predetermined amount of time, then the ventilator 100 will deliver a breath at the expiration of the predetermined amount of time. In some embodiments, the predetermined amount of time, such as but not limited to 20 seconds, 30 seconds, or 60 seconds of exhalation time, starts to elapse upon the delivery of a breath. The predetermined amount of time may be input by the clinician or calculated by the ventilator.

In another embodiment, the second trigger condition is met when the patient 150 reaches a stable portion of exhalation as determined by the backup trigger module 115. In order to determine a stable portion of exhalation, the ventilator 100 monitors the estimated exhalation flow. In some embodiments, the backup trigger module 115 collects multiple exhalation flow estimates for a set period during exhalation after the expiration of a restricted period. The restricted period as used herein is a predetermined time period that starts at the beginning of exhalation. The patient 150 is prevented from triggering ventilation during the predetermined time period of the restricted period. For example, the restricted period may be 25 ms, 50 ms, 100 ms, 200 ms, and/or any other suitable time period for preventing the patient 150 from triggering inspiration. In other embodiments, the backup trigger module 115 determines a stable portion of exhalation without utilizing a restricted period. In an embodiment, the backup trigger module 115 determines stability by monitoring the estimated exhalation flow every computation cycle. In some embodiments, the computational cycle is every 5 ms. If the difference between two successive exhalation flow estimates is zero, or about zero, then a stable portion of exhalation has been determined and the backup trigger module 115 will instruct the ventilator 100 to deliver a breath. In an additional embodiment, the second trigger condition is met when the patient 150 after reaching a stable portion of exhalation detects a negative change in base flow as determined by the backup trigger module 115.

In another embodiment, the trigger modules 113, 115 utilize a counter relating to pressure measurements, herein referred to as a pressure slope counter, as an inspiration trigger threshold, or second trigger condition. A pressure slope may be calculated in a variety of ways, such as based on the difference between a previous pressure measurement and a current pressure measurement, such as but not limited to an inspiratory pressure measurement. For example, the pressure slope is calculated as the previous pressure measurement subtracted from the current pressure measurement. Further, if the pressure slope is less than zero, the pressure slope counter will be incremented by one. In an embodiment, the inspiration trigger threshold is met and/or exceeded when the pressure slope counter is greater than or equal to one. The pressure slope counter may be reset to zero after an inspiration trigger is detected, or at another appropriate time. Any suitable use of a pressure slope counter may be utilized by the trigger modules 113, 115 for triggering an inspiration. For example, in some embodiments, the trigger threshold is any pressure slope counter value greater than one.

In some embodiments, the trigger modules 113, 115 utilize a change in flow rate as an inspiration trigger threshold. For example, the inspiration trigger threshold may be a change in flow rate of −1.5 liters per minute (LPM), −2 LPM, −3 LPM, −4 LPM, −5 LPM, −6 LPM, −7 LPM, and −8 LPM or may be a range of a change in flow rate, such as a range of −3 LPM to −6 LPM or −4 LPM to −7 LPM. This list is exemplary only and is not meant to be limiting. Any suitable changes in flow rate may be utilized by the trigger modules 113, 115 for triggering an inspiration. For example, in some embodiments, the trigger threshold is any detected drop in flow rate that is at least 1.5 LPM.

The controller 110 is operatively coupled with the pneumatic system 102, signal measurement and acquisition systems such as but not limited to a plurality of sensors 107, and an operator interface 120 that may enable an operator to interact with the ventilator 100 (e.g., change ventilator settings, select operational modes, view monitored parameters, etc.).

In some embodiments, the controller 110 includes memory 112, one or more processors 116, storage 114, and/or other components of the type commonly found in command and control computing devices, as illustrated in FIG. 1A. In alternative embodiments, the controller 110 is a separate component from the operator interface 120 and pneumatic system 102. In other embodiments, the controller 110 is located in other components of the ventilator 100, such as in the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Further, the controller 110 determines if there is a malfunction that makes exhalation flow undeterminable and/or unreliable. Accordingly, the controller 110 determines if the exhalation flow sensor 111a, exhalation pressure sensor 111b, and/or the valve command (i.e., the main driver 103) of the exhalation valve 123 are unreliable. If the exhalation flow sensor 111a, exhalation pressure sensor 111b, and/or the valve command are determined to be unreliable by the controller 110, then the monitored expiratory flow, monitored expiratory pressure, valve position, valve current, valve current command, valve dampening command, and etc. may all be unreliable.

Several different systems and methods are currently utilized and known in the art for determining a malfunction in the exhalation module 108 and components of the exhalation module (e.g., exhalation flow sensor 111a, exhalation pressure sensor 111b, exhalation valve 123). The controller 110 detects a malfunction utilizing any of these known systems or methods. For example, malfunctions may be detected based on changes in voltages, temperatures, wattages, coefficients, humidity, and/or overcurrent for various components (e.g., exhalation flow sensor, exhalation valve) of the exhalation module 108.

If the controller 110 determines a malfunction, the controller 110 switches from, or instructs a switch from, the main trigger module 113 and the main driver 103 to the backup trigger module 115 and the backup driver 105. In an embodiment, the backup trigger module 115 activates the flow estimation module 117 which estimates the exhalation flow based on the monitored inspiratory pressure and/or monitored inspiratory flow. In some embodiments, the ventilator 100, unlike prior art, is able to maintain a spontaneous breath mode of ventilation. During the spontaneous breath mode of ventilation the inspiratory triggering is based on an estimated exhalation flow instead of a monitored exhalation flow. Further, the controller 110 instructs the pneumatic system 102 to deliver an EBUV mode of ventilation. The EBUV mode is a spontaneous mode of ventilation. The pressure to be administered to the patient 150 during inspiration and exhalation of the spontaneous breath is determined by the ventilator 100. Further, the inspiratory time, and respiratory rate for the patient 150 are also determined by the ventilator 100. These variables determine the pressure of the gas delivered to the patient 150 during each spontaneous breath inspiration and exhalation. For the EBUV mode, when the inspiratory time is equal to the prescribed inspiratory time, the ventilator 100 initiates exhalation. Exhalation lasts from the end of inspiration until an inspiratory trigger is detected or until the expiration of a predetermined amount of time. Upon the detection of an inspiratory trigger, another spontaneous breath is given to the patient 150.

During an EBUV mode, the ventilator 100 maintains the same pressure waveform at the mouth, regardless of variations in lung or airway characteristics, e.g., respiratory compliance and/or respiratory resistance. However, the volume and flow waveforms may fluctuate based on lung and airway characteristics. In some embodiments, the ventilator 100 determines the set pressure (including the inspiratory pressure and the PEEP), the inspiratory time, and respiration rate based on known ventilator parameters that have not been corrupted by the determined malfunction, such as weight, height, sex, age, and disease state. In other embodiments, the set pressure (including the inspiratory pressure and the PEEP), the inspiratory time, and the respiration rate are predetermined by the ventilator 100 upon the detection of a malfunction and are the same for any patient 150 being ventilated by the ventilator 100.

If the controller 110 does not determine a malfunction, the controller 110 does not change to the backup driver 105 and backup trigger module 115 and continues to deliver ventilation utilizing the main driver 103 and to trigger an inspiration utilizing the main trigger module 113. In some embodiments, if the controller 110 determines a malfunction the controller 110 switches to the backup driver 105 to control ventilation to the patient and to the backup trigger module 115 to trigger inspiration from the main driver 103 and the main trigger module 113. In some embodiments, the controller 110 is part of the exhalation module 108. In some embodiments, the controller 110 is part of the pneumatic system 102. In other embodiments, the controller 110 is a module separate from the pneumatic system 102.

The ventilator 100 also includes a plurality of sensors 107 communicatively coupled to the ventilator 100. The sensors 107 may be located in the pneumatic system 102, ventilation tubing system 130, and/or on the patient 150. The embodiment of FIG. 1A illustrates a plurality of sensors 107 in pneumatic system 102.

The sensors 107 may communicate with various components of the ventilator 100, e.g., the pneumatic system 102, other sensors 107, the exhalation module 108, the inspiratory module 104, a processor 116, the controller 110, and any other suitable components and/or modules. In one embodiment, the sensors 107 generate output and send this output to the pneumatic system 102, other sensors 107, the exhalation module 108, the inspiratory module 104, the processor 116, the controller 110, the main trigger module 113, the backup trigger module 115, the flow estimation module 117, and any other suitable components and/or modules.

The sensors 107 may employ any suitable sensory or derivative technique for monitoring one or more patient parameters or ventilator parameters associated with the ventilation of a patient 150. The sensors 107 may detect changes in patient parameters indicative of patient inspiratory or exhalation triggering effort, for example. The sensors 107 may be placed in any suitable location, e.g., within the ventilatory circuitry or other devices communicatively coupled to the ventilator 100. Further, the sensors 107 may be placed in any suitable internal location, such as, within the ventilatory circuitry or within components or modules of the ventilator 100. For example, the sensors 107 may be coupled to the inspiratory and/or exhalation modules 104, 108 for detecting changes in, for example, inspiratory flow, inspiratory pressure, expiratory pressure, and expiratory flow. In other examples, the sensors 107 may be affixed to the ventilatory tubing 130 or may be embedded in the tubing itself. According to some embodiments, the sensors 107 may be provided at or near the lungs (or diaphragm) for detecting a pressure in the lungs. Additionally or alternatively, the sensors 107 may be affixed or embedded in or near the wye-fitting 170 and/or patient interface 180. Any sensory device useful for monitoring changes in measurable parameters during ventilatory treatment may be employed in accordance with embodiments described herein.

For example, in some embodiments, the one or more sensors 107 of the ventilator 100 include an inspiratory flow sensor 109a and/or an exhalation flow sensor 111a as illustrated in FIG. 1B. In one embodiment, the inspiratory flow sensor 109a is located in the inspiratory limb 132 and is controlled by the inspiratory module 104 and/or the flow estimation module 117. However, the inspiratory flow sensor 109a may be located in any suitable position for monitoring inspiratory flow and may be monitored by any suitable ventilator component, such as the pressure generating system 102. In one embodiment, the exhalation flow sensor 111a is located in the exhalation limb 134 and is monitored by the exhalation module 108 and/or the controller 110, including the main trigger module 113. However, the exhalation flow sensor 111a may be located in any suitable position for monitoring exhalation flow and may be monitored by any suitable ventilator component, such as the pressure generating system 102.

Further, in some embodiments, the one or more sensors 107 of the ventilator 100 also include an inspiratory pressure sensor 109b and/or an exhalation pressure sensor 111b as illustrated in FIG. 1B. In one embodiment, the inspiratory pressure sensor 109b is located in the inspiratory limb 132 and is controlled by the inspiratory module 104 and the flow estimation module 117. However, the inspiratory pressure sensor 109b may be located in any suitable position for monitoring inspiratory pressure and may be monitored by any suitable ventilator component, such as the pressure generating system 102. In one embodiment, the exhalation pressure sensor 111b is located in the exhalation limb 134 and is monitored by the exhalation module 108 and/or the controller 110. However, the exhalation pressure sensor 111b may be located in any suitable position for monitoring exhalation pressure and may be monitored by any suitable ventilator component, such as the pressure generating system 102.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated by derivation according to the Equation of Motion or other known relationships. For example, in some embodiments, inspiration flow is derived from measured inspiration pressure and vice versa. In another example, exhalation pressure is derived from exhalation flow and vice versa. Accordingly, the terms “exhalation flow” and “exhalation pressure”, while having different meanings, are utilized interchangeably herein. Therefore, the term “exhalation flow” encompasses the term “exhalation pressure” and the term “exhalation pressure” encompasses “exhalation flow.”

The pneumatic system 102 may include a variety of other components, including mixing modules, valves, tubing, accumulators 124, filters, etc. For example, FIG. 1B illustrates the use of an accumulator 124.

In one embodiment, the operator interface 120 of the ventilator 100 includes a display 122 communicatively coupled to the ventilator 100. The display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, the operator interface 120 may accept commands and input through the display 122.

The display 122 may also provide useful information in the form of various ventilatory data regarding the physical condition of the patient 150. The useful information may be derived by the ventilator 100, based on data collected by the processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In some embodiments, the display 122 may illustrate the use of an EBUV mode during a malfunction and/or any other information known, received, or stored by the ventilator 100, such as the estimated exhalation flow, the net flow, the predetermined flow deviation trigger threshold, and/or a display representative of the time left before expiration of the predetermined amount of time.

FIG. 2 illustrates an embodiment of a method 200 for triggering inspiration during ventilation of a patient on a ventilator. Further, the method 200 provides ventilation after a malfunction is detected that prevents the exhalation flow from being determined and/or reliable. The ventilation provided after a malfunction is referred to herein as an exhalation backup-ventilation mode (EBUV). The method 200 begins at the start of ventilation.

As illustrated, the method 200 includes a fixed base flow delivery operation 202. During the fixed base flow delivery operation 202, the ventilator delivers a fixed base flow. The fixed base flow is a continuous flow of gas through the ventilation tubing system from the inspiratory limb through the exhalation limb. This continuous flow allows the ventilator to trigger inspiration as well as determine the phase of breath (i.e. inhalation, exhalation, or between breaths) the patient is currently in. For example, if the flow through the exhalation limb is equal and opposite of the flow through the inspiratory limb, then the ventilator determines that the patient is currently between breaths as there is no flow into or out of the lungs of the patient. In a further example, if the flow through the inspiratory limb exceeds the flow through the exhalation limb, then the ventilator determines that the patient is inhaling and the flow of gas is going into the patient's lungs. In yet a further example, if the flow through the exhalation limb exceeds the flow through the inspiratory limb, then the ventilator determines that the patient is exhaling and the flow of gas is flowing from the patient's lungs (and inspiratory limb) through the exhalation limb.

Further, the method 200 includes a monitoring operation 204. During the monitoring operation 204, the ventilator monitors ventilator parameters. In some embodiments, the ventilator during the monitoring operation 204 monitors numerous ventilator parameters. As used herein ventilator parameters include any parameter that may be monitored by the ventilator. In an embodiment, the ventilator during the monitoring operation 204 monitors inspiratory flow, inspiratory pressure, and exhalation flow. Sensors suitable for this detection may include any suitable sensing device as known by a person of skill in the art for a ventilator, such as an inspiratory flow sensor, inspiratory pressure sensor, and an exhalation flow sensor. In an embodiment, the ventilator during the monitoring operation 204 delivers ventilation based at least on the monitored exhalation flow.

The method 200 further includes a malfunction decision operation 206. During the malfunction decision operation 206, the ventilator determines a malfunction that makes the monitored exhalation flow unreliable. The ventilator during the malfunction decision operation 206 determines a malfunction by determining if the exhalation flow sensor, exhalation pressure sensor, exhalation valve command (i.e. main driver), exhalation module, and/or any other sensor and/or module relevant to exhalation flow are unreliable. If the exhalation flow sensor, exhalation pressure sensor, exhalation valve command, exhalation module, and/or any other sensor and/or module relevant to exhalation flow are determined to be unreliable by the ventilator during the malfunction decision operation 206, then the monitored exhalation flow, monitored exhalation pressure, and etc. may all be unreliable.

The ventilator during malfunction decision operation 206 detects a malfunction that may make ventilator parameters undeterminable or unreliable. Several different systems and methods are currently utilized and known in the art for determining a malfunction in the exhalation limb and components of the exhalation module (e.g., exhalation flow sensor, exhalation pressure sensor, exhalation valve). The ventilator during the malfunction decision operation 206 detects a malfunction utilizing any of these known systems or methods. For example, malfunctions may be detected based on changes in voltages, temperatures, wattages, coefficients, humidity, and/or overcurrent for various components (e.g., exhalation flow sensor, exhalation valve) of the exhalation module. In an embodiment, during the malfunction decision operation 206 if the ventilator determines a malfunction, the ventilator displays information relating to the malfunction or to the EBUV mode such as but not limited to an indicator that displays the use of an EBUV mode, an estimated exhalation flow value, a flow deviation, a flow deviation trigger threshold, a trigger threshold, a predetermined amount of time used as a trigger threshold, and/or an indicator that displays the presence of a malfunction.

If the ventilator during the malfunction decision operation 206 determines a malfunction is present, the ventilator selects to perform an estimation operation 210. If the ventilator during the malfunction decision operation 206 does not determine a malfunction, the ventilator selects to perform a monitored trigger detection operation 208.

The method 200 includes a monitored trigger detection operation 208. The ventilator during the monitored trigger detection operation 208 determines if a first inspiratory trigger is detected. The first inspiratory trigger is detected when a monitored patient and/or ventilator parameter exceeds, or breaches, an inspiratory trigger threshold. In some embodiments, the inspiratory trigger threshold is received from operator input. In other embodiments, the inspiratory trigger threshold is based on ventilator and/or patient parameters. In some embodiments, a net negative change in flow rate below a delivered base flow is the inspiratory trigger threshold. For example, the inspiratory trigger threshold may be a change in flow rate of −1.5 LPM, −2 LPM, −3 LPM, −4 LPM, −5 LPM, −6 LPM, −7 LPM, and −8 LPM or may be a range of a change in flow rate, such as a range of −3 LPM to −6 LPM or −4 LPM to −7 LPM. This list is exemplary only and is not meant to be limiting. Any suitable change in flow rate below the delivered base flow may be utilized by the ventilator as the inspiratory trigger threshold during the monitored trigger detection operation 208. In an embodiment, a known fixed base flow and a monitored exhalation flow are combined, such as arithmetically, to determine a first net flow, or first flow deviation, that is compared against the inspiratory trigger threshold. A slight drop in the base flow through the exhalation module during exhalation may indicate that a patient is attempting to inspire. A drop in base flow is attributable to a redirection of gases into the patient's lungs (in response to a slightly negative pressure gradient).

In another embodiment, the first trigger condition is met when the patient reaches a stable portion of exhalation as determined by the ventilator during the monitored trigger detection operation 208. In order to determine a stable portion of exhalation the ventilator monitors exhalation flow and/or exhalation pressure. In some embodiments, the ventilator during the monitored trigger detection operation 208 collects multiple exhalation flow and/or exhalation pressure readings for a set period during exhalation after the expiration of a restricted period. The restricted period as used herein is a predetermined time period that starts at the beginning of exhalation. The patient is prevented from triggering ventilation during the predetermined time period of the restricted period. For example, the restricted period may be 25 ms, 50 ms, 100 ms, 200 ms, and/or any other suitable time period for preventing the patient from triggering inspiration. In an embodiment, the ventilator during the monitored trigger detection operation 208 determines stability by monitoring the exhalation flow every computation cycle. In some embodiments, the computational cycle is every 5 ms. If the difference between two successive exhalation flow readings is zero, or near zero, then a stable portion of exhalation has been determined and the ventilator selects to perform a delivery operation 216.

In another embodiment, the first trigger condition utilizes a pressure slope counter relating to pressure measurements as determined by the ventilator during the monitored trigger detection operation 208. A pressure slope may be calculated in a variety of ways, such as based on the difference between a previous pressure measurement and a current pressure measurement, such as but not limited to an inspiratory pressure measurement. For example, the pressure slope is calculated as the previous pressure measurement subtracted from the current pressure measurement. Further, if the pressure slope is less than zero, the pressure slope counter will be incremented by one. In an embodiment, the first trigger condition is met when the pressure slope counter is greater than or equal to one. The pressure slope counter may be reset to zero after an inspiration trigger is detected, or at another appropriate time. Any suitable use of a pressure slope counter may be utilized by the ventilator during the monitored trigger detection operation 208 for triggering an inspiration. For example, in some embodiments, the first trigger condition is met when the pressure slope counter has a value greater than one.

In an embodiment, if the ventilator during the monitored trigger detection operation 208 determines that ventilator and/or patient parameters meet and/or exceed the inspiratory trigger threshold, or first trigger condition, during exhalation, the ventilator selects to perform a delivery operation 216. If the ventilator during the monitored trigger detection operation 208 determines that ventilator and/or patient parameters do not meet and/or exceed the inspiratory trigger threshold during exhalation, or the first trigger condition, the ventilator continues to monitor the ventilator and/or patient parameters and compare them to the trigger threshold until the ventilator and/or patient parameters meet and/or exceed the trigger threshold, the first trigger condition, or until the expiration of a predetermined amount of time.

In one embodiment, the ventilator is preconfigured to select to perform a delivery operation 216 after a predetermined amount of exhalation time to prevent the patient from becoming under-ventilated. Accordingly, the predetermined amount of exhalation time (e.g., known as an apnea interval in some ventilators) is the trigger threshold, or the first trigger condition, in this embodiment. For example, the ventilator during the monitored trigger detection operation 208 will automatically select to perform a delivery operation 216 after 20 seconds, 30 seconds, or 60 seconds of exhalation time. In some embodiments, the predetermined amount of time may be input by the clinician or calculated by the ventilator. In some embodiments, the predetermined amount of time is determined by the clinician and/or ventilator based on whether the patient is an infant, child, adult, male, female, and/or suffering from a specific disease state.

The method 200 includes the estimation operation 210. The ventilator during the estimation operation 210 determines an estimated exhalation flow or updates a previously calculated estimated exhalation flow with a more current estimated exhalation flow. The exhalation flow is estimated because the ventilator is not able to determine a reliable exhalation flow if the exhalation flow sensor and/or exhalation flow module are malfunctioning. In another example, the ventilator may not be able to determine the exhalation flow if the ventilator does not contain an exhalation flow sensor. For example, the ventilator cannot determine the exhalation flow during EBUV. The estimated exhalation flow is determined based on the monitored inspiratory pressure and/or inspiratory flow. In an embodiment, the ventilator during the estimation operation 210 ceases ventilation based on the monitored exhalation flow. In an embodiment, the ventilator during the estimation operation 210 delivers ventilation based at least on the estimated exhalation flow. Once the estimated exhalation flow is determined the ventilator selects to perform an estimated trigger detection operation 212.

The method 200 includes the estimated trigger detection operation 212. The ventilator during the estimated trigger detection operation 212 determines if a second inspiratory trigger is detected. The second inspiratory trigger is detected when an estimated patient and/or ventilator parameter exceeds, or breaches, an inspiratory trigger threshold. In some embodiments, the inspiratory trigger threshold is received from operator input. In other embodiments, the inspiratory trigger threshold is based on ventilator and/or patient parameters. In some embodiments, a net negative change in flow rate below a delivered base flow is the inspiratory trigger threshold. For example, the inspiratory trigger threshold may be a change in flow rate of −1.5 LPM, −2 LPM, −3 LPM, −4 LPM, −5 LPM, −6 LPM, −7 LPM, and −8 LPM or may be a range of a change in flow rate, such as a range of −3 LPM to −6 LPM or −4 LPM to −7 LPM. This list is exemplary only and is not meant to be limiting. Any suitable change in flow rate below the delivered base flow may be utilized by the ventilator as the inspiratory trigger threshold during the estimated trigger detection operation 212. Because the monitored exhalation flow is undeterminable or unreliable, the estimated exhalation flow is combined with a known fixed base flow, such as arithmetically, to determine a second net flow, or second flow deviation, that is compared against the inspiratory trigger threshold. A slight drop in the base flow through the exhalation module during exhalation may indicate that a patient is attempting to inspire. A drop in base flow is attributable to a redirection of gases into the patient's lungs (in response to a slightly negative pressure gradient).

In another embodiment, the second trigger condition is met when the patient reaches a stable portion of exhalation as determined by the ventilator during the estimated trigger detection operation 212. In order to determine a stable portion of exhalation the ventilator during the estimated trigger detection operation 212 monitors the estimated exhalation flow. In some embodiments, the ventilator during the estimated trigger detection operation 212 collects multiple exhalation flow estimates for a set period during exhalation after the expiration of a restricted period. The restricted period as used herein is a predetermined time period that starts at the beginning of exhalation. The patient is prevented from triggering ventilation during the predetermined time period of the restricted period. For example, the restricted period may be 25 ms, 50 ms, 100 ms, 200 ms, and/or any other suitable time period for preventing the patient from triggering inspiration. In an embodiment, the ventilator during the estimated trigger detection operation 212 determines stability by monitoring the estimated exhalation flow every computation cycle. In some embodiments, the computational cycle is every 5 ms. If the difference between two successive exhalation flow estimates is zero, or near zero, then a stable portion of exhalation has been determined and the ventilator selects to perform a delivery operation 216.

In another embodiment, the second trigger condition utilizes a pressure slope counter relating to pressure measurements as determined by the ventilator during the estimated trigger detection operation 212. A pressure slope may be calculated in a variety of ways, such as based on the difference between a previous pressure measurement and a current pressure measurement, such as but not limited to an inspiratory pressure measurement. For example, the pressure slope is calculated as the previous pressure measurement subtracted from the current pressure measurement. Further, if the pressure slope is less than zero, the pressure slope counter will be incremented by one. In an embodiment, the second trigger condition is met when the pressure slope counter is greater than or equal to one. The pressure slope counter may be reset to zero after an inspiration trigger is detected, or at another appropriate time. Any suitable use of a pressure slope counter may be utilized by the ventilator during the estimated trigger detection operation 212 for triggering an inspiration. For example, in some embodiments, the second trigger condition is met when the pressure slope counter has a value greater than one.

In an embodiment, if the ventilator during the estimated trigger detection operation 212 determines that ventilator and/or patient parameters meet and/or exceed the inspiratory trigger threshold during exhalation, or the second trigger condition, the ventilator selects to perform a delivery operation 216. If the ventilator during the estimated trigger detection operation 212 determines that ventilator and/or patient parameters do not meet and/or exceed the inspiratory trigger threshold during exhalation, or the second trigger condition, the ventilator continues to monitor the ventilator and/or patient parameters and compare them to the trigger threshold until the ventilator and/or patient parameters meet and/or exceed the trigger threshold, the second trigger condition, or until the expiration of a predetermined amount of time.

In one embodiment, the ventilator is preconfigured to select to perform a delivery operation 216 after a predetermined amount of exhalation time to prevent the patient from becoming under-ventilated. Accordingly, the predetermined amount of exhalation time is the trigger threshold, or second trigger condition, in this embodiment. For example, the ventilator during the estimated trigger detection operation 212 will automatically select to perform a delivery operation 216 after 20 seconds, 30 seconds, or 60 seconds of exhalation time. In some embodiments, the predetermined amount of time may be input by the clinician or calculated by the ventilator. In some embodiments, the predetermined amount of time is determined by the clinician and/or ventilator based on whether the patient is an infant, child, adult, male, female, and/or suffering from a specific disease state.

Further, the method 200 includes the delivery operation 216. The ventilator during the delivery operation 216 delivers a next breath to the patient. The breath delivered to the patient may be determined by the ventilator and/or patient parameters. For example, the delivered breath may be based on a selected breath type or ventilation mode, such as EBUV. After the breath is delivered to the patient, the ventilator selects to return to the monitoring operation 204.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

What is claimed is:

1. A method for ventilating a patient with a ventilator, comprising:
delivering a fixed base flow;
monitoring inspiratory pressure and exhalation flow during ventilation of a patient with a ventilator;
delivering ventilation based at least on the monitored exhalation flow;
triggering inspiration during the ventilation based at least on the monitored exhalation flow based on the first of at least one of the following events to occur:
detecting a first trigger condition; and
detecting expiration of a predetermined amount of time;
determining a malfunction that makes the monitored exhalation flow unreliable; and in response to the malfunction:
ceasing ventilation based on the monitored exhalation flow;
estimating an exhalation flow based on the monitored inspiratory pressure;
delivering ventilation based at least on the estimated exhalation flow;
triggering inspiration during the ventilation based at least on the estimated exhalation flow based on the first of at least one of the following events to occur:
detecting a second trigger condition based at least on the estimated exhalation flow; and
detecting expiration of the predetermined amount of time.

2. The method of claim 1, wherein the second trigger condition is a slope of the estimated flow signal at about zero.

3. The method of claim 1, wherein the second trigger condition is a slope of the estimated flow signal that after reaching about zero decreases below zero.

4. The method of claim 1, wherein the second trigger condition is a flow deviation based on the fixed base flow and the estimated expiratory flow that breaches a trigger threshold.

5. The method of claim 4, wherein the trigger threshold is a drop in flow rate of at least 1.5 LPM.

6. The method of claim 4, wherein the trigger threshold is a range of a change in flow rate of −3 LPM to −6 LPM.

7. The method of claim 1, wherein the second trigger condition is a pressure slope counter value greater than or equal to 1.

8. The method of claim 1, wherein the second trigger condition is a pressure slope less than zero.

9. The method of claim 1, wherein the predetermined amount of time is about 20 seconds of exhalation time.

10. The method of claim 1, wherein the malfunction is at least one of a malfunctioning exhalation flow sensor, main driver, exhalation module, and exhalation pressure sensor.

11. The method of claim 1, wherein estimating an exhalation flow further includes inputting the monitored inspiratory pressure into an estimated exhalation flow algorithm.

12. The method of claim 1, further comprising:
in response to the malfunction:
displaying at least one of a use of a spontaneous exhalation backup ventilation mode, a flow deviation, the estimated exhalation flow, a trigger threshold, and the predetermined amount of time.

13. A ventilator system comprising:
a pressure generating system adapted to generate a flow of breathing gas including a fixed base flow;
a ventilation tubing system including a patient interface for connecting the pressure generating system to a patient;
an exhalation valve connected to the ventilation tubing system;
a plurality of sensors operatively coupled to at least one of the pressure generating system, the patient, and the ventilation tubing system for monitoring inspiratory pressure, inspiratory flow, exhalation pressure, and exhalation flow;
an exhalation flow estimation module, the exhalation flow estimation module estimates exhalation flow based on the monitored inspiratory pressure;
a main driver, the main driver controls the exhalation valve to deliver ventilation to the patient based at least on at least one of the exhalation pressure and the exhalation flow monitored by the plurality of sensors;
a main trigger module, the main trigger module triggers an inspiration based on the first of at least one of the following events to occur:
detection of a first trigger condition, and
expiration of a predetermined amount of time;
a backup driver, the backup driver controls the exhalation valve to deliver the ventilation to the patient based on at least one of the inhalation pressure and the inhalation flow monitored by the plurality of sensors;
a backup trigger module, the backup trigger module triggers the inspiration based on the first of at least one of the following events to occur:
detection of a second trigger condition based at least on the estimated exhalation flow, and
expiration of the predetermined amount of time;
a controller, the controller determines a malfunction that makes the monitored exhalation flow unreliable and switches from the main driver and the main trigger module to the backup driver and the backup trigger module.

14. The ventilator system of claim 13, further comprising:
a display that displays at least one of a use of a spontaneous exhalation backup ventilation mode, a flow deviation, the estimated exhalation flow, a trigger threshold, and the predetermined amount of time.

15. The ventilator system of claim 13, wherein the controller detects the malfunction in at least one of an exhalation flow sensor, a main driver, and an exhalation pressure sensor.

16. The ventilator system of claim 13, wherein the backup driver is on a circuit isolated from the main driver.

17. The ventilator system of claim 13, wherein the second trigger condition is a slope of the estimated flow signal at about zero.

18. The ventilator system of claim 13, wherein the second trigger condition is a pressure slope counter value greater than or equal to 1.

19. The ventilator system of claim 13, wherein the second trigger condition is a flow deviation based on the fixed base flow and the estimated expiratory flow that breaches a trigger threshold.

20. A computer-readable medium having computer-executable instructions for performing a method of ventilating a patient with a ventilator, the method comprising:
repeatedly delivering a fixed base flow;
repeatedly monitoring inspiratory pressure and exhalation flow during ventilation of a patient with a ventilator;
repeatedly delivering ventilation based at least on the monitored exhalation flow;
repeatedly triggering inspiration during the ventilation based at least on the monitored exhalation flow based on the first of at least one of the following events to occur:
detecting a first trigger condition; and
detecting expiration of a predetermined amount of time;
determining a malfunction that makes the monitored exhalation flow unreliable; and in response to the malfunction:
ceasing ventilation based on the monitored exhalation flow;
repeatedly estimating an exhalation flow based on the monitored inspiratory pressure;
repeatedly delivering ventilation based at least on the estimated exhalation flow;
repeatedly triggering inspiration during the ventilation based at least on the estimated exhalation flow based on the first of at least one of the following events to occur:
detecting a second trigger condition based at least on the estimated exhalation flow; and
detecting expiration of the predetermined amount of time.
